(19) United States
(12) Patent Application Publication
(10) Pub. No.: US 2013/0035675 A1
(43) Pub. Date: Feb. 7, 2013

(54) NON-UNIFORM BEAM PHOTOTHERAPEUTIC DOSAGE DETERMINATION METHOD

(71) Applicants: Mirko Georgiev Mirkov, Chelmsford, MA (US); Rafael Armando Sierra, Palmer, MA (US); George E.S. Cho, Hopkinton, MA (US); James Henry Boll, Newton, MA (US)

(72) Inventors: Mirko Georgiev Mirkov, Chelmsford, MA (US); Rafael Armando Sierra, Palmer, MA (US); George E.S. Cho, Hopkinton, MA (US); James Henry Boll, Newton, MA (US)

(73) Assignee: CYNOSURE, INC., Westford, MA (US)

(21) Appl. No.: 13/566,306

(22) Filed: Aug. 3, 2012

Related U.S. Application Data
(60) Provisional application No. 61/515,102, filed on Aug. 4, 2011.

(51) Int. Cl. A61B 18/20

(52) U.S. Cl. 606/9

(57) ABSTRACT

This application provides a consumer device for aesthetic applications, and methods for titrating doses of therapeutic light output from the device in the form of a non-uniform beam, in connection with dermal rejuvenation and cosmetic applications.

### CROSS-REFERENCE TO RELATED APPLICATION

This application claims the benefit of U.S. Provisional Patent application Ser. No. 61/515,102, entitled “Non-Uniform Beam Phototherapeutic Dosage Determination Method,” which was filed Aug. 4, 2011. The entirety of the aforementioned application is herein incorporated by reference.

### FIELD OF THE INVENTION

This application relates to the field of consumer devices for aesthetic applications. Particularly, disclosed herein are methods for titrating doses of therapeutic light output from the device in the form of a non-uniform beam, in connection with dermal rejuvenation and cosmetic applications.

### BACKGROUND

Sun exposure results in numerous changes to the skin over time. These changes reduce the elasticity and uniform color and tone of skin and are collectively referred to as photoaging, which are exemplified by the manifestation of wrinkles and lines, sagging and various discolorations. One cosmetically undesirable aspect of skin photoaging manifests as pigmentary changes in the skin, most often uneven pigmentation commonly referred to as “age spots”. Skin pigmentation is the result of melanocytes residing at the dermal-epidermal interface expressing the pigment melanin. Uneven skin pigmentation is associated either with uneven concentration of melanocytes or uneven production of melanin by melanocytes. Another cosmetically undesirable aspect of skin photoaging manifests as wrinkles and lines, and loss of skin tone.

Current light based treatments for addressing uneven skin pigmentation use lasers or intense pulse lights (IPL). Although such treatments are effective, multiple treatments are required to achieve optimal results, with the associated costs of repeat office visits and associated pain from the procedure(s). In addition such treatment requires sophisticated feathering techniques in order to avoid sharp boundaries between treated and untreated skin and the inadvertent creation of regions of uneven pigmentation.

Current light based treatments for firming and tightening skin in order to reduce the visible appearance of wrinkles and sagging employs lasers, to heat treatment regions to relatively high temperatures to melt collagen, which renatures and tightens the treatment region. However, to achieve collagen restructuring, the treatment zone is subjected to temperatures that are destroy cells in the beam area. There is pain and post-treatment discomfort from the procedure, which is undesirable.

Accordingly, there remains a need in the art for improved dermal rejuvenation devices and methods, that improves skin tone and coloration, and smoothes fine lines and wrinkles.

### BRIEF DESCRIPTION OF THE FIGURES

FIG. 1 provides the range of combinations of power settings (W) and pulse width values (ms) used in the initial Phase One study.

FIG. 2 is a patient photo immediately following the final of 20 weekday exposures provided over the 4 week treatment period.

FIG. 3 illustrates that higher power settings and longer pulse durations drive effect/sensation.

FIG. 4 illustrates various power and time combinations, and patient responses affirming detection of sensation from the treatment.

FIG. 5 shows that Mexameter readings correlate to visual observations.

FIG. 6 illustrates that Mexameter readings over the duration of the study indicate the therapeutic response is cumulative.

FIG. 7 show that energy and exposure duration drive clinical effect.

FIG. 8 shows a plot of power versus pulse duration, which was used to derive the power and time settings used for the Phase Two study.

FIG. 9 shows the prevalence of visible erythema immediately following treatment

FIG. 10 illustrates that as detected by Mexameter, the intensity of erythema decreases over the course of twenty treatments.

FIG. 11 illustrates, that at two time points, the two different power settings show no significant difference in resultant erythema.

FIG. 12 demonstrates the respective average drop in melanin scores as recorded by Mexameter.

FIG. 13 shows, on average, patients rated the sensation immediately following treatment to be a 1 (out of 10) in the SUB area and a 3 (out of 10) in the SUPRA area.

FIG. 14 illustrates Expert Grader scoring at baseline, end of treatment and following 6-week regression in both SUB and SUPRA treatment areas.

FIG. 15 shows Primos analysis of Replica molds taken in peri-orbital and middle cheek areas.

FIG. 16 shows Sample Primos images for one exemplary subject.

FIG. 17 illustrates improvements to photodamage and fine lines/wrinkles were recorded on the cheek, under eye and crow's feet areas in both SUB and SUPRA regions.

FIG. 18 illustrates several small foci of parakeratosis in the stratum corneum in almost all of the Day 1 and Day 3 SUPRA samples.

FIG. 19 shows procollagen production increases up to Day 7 following 20 laser exposures.

### DETAILED DESCRIPTION

We describe herein, a device for aesthetic treatment of photodamaged skin in a human, and methods for titrating doses of therapeutic light output from the device in the form of a non-uniform beam, in connection with dermal rejuvenation and cosmetic applications. The result of such treatments include improvements in the tone and elasticity of skin, reduction of fine lines and wrinkles, and more uniform pigmentation, such as the reduction or elimination of age spots, vascular lesions, birthmarks and the like.

A non-uniform output beam is delivered from a source of light as described in our related applications U.S. Ser. No. 11/347,672; 12/635,295; 12/947,310, and PCT/US10/026432. The non-uniform beam is characterized by a cross-section corresponding to an array of relatively small, relatively high-intensity, spaced-apart central regions superimposed on a relatively large, and relatively low-intensity background region. Operatively, this produces within the area of the beam, relatively hotter regions and relatively cooler regions. This non-uniform beam provides for unique physiological effects as compared to standard uniform output laser beams that demonstrate relative uniform energy output across the planar surface of the beam. Such effects are related to the fluence and duration of the light pulse, and include various quantifiable physiological effects. Exemplary temperature dependent effects include but are not limited to parakeratosis, perivascular mononuclear infiltration, keratinocyte necrosis, collagen denaturation, and procollagen expression in dermal cells. Other cellular markers (e.g., nucleic acids and proteins) are useful in detecting more subtle responses of skin to less aggressive treatments, and are discussed in more detail below.

Various combinations of wavelength, power, spot size, treatment duration and recovery intervals are possible, and the particular combination is selected based on the desired therapeutic effect. For example, in treating age spots and pigmentation a device wavelength is chosen to be preferentially absorbed by melanin (between 400 nm and 1400 nm, and more preferably 500 nm to 1100 nm). Accordingly, an exemplary device for such purposes has a wavelength of about 800 nm, a pulse duration of about 5 ms and an overall treatment area of 1 cm² that is output as a non-uniform beam characterized by a cross-section corresponding to an array of relatively small, relatively high intensity, spaced-apart central regions superimposed on a relatively large, relatively low intensity background region. If such exemplary device has an output power of about 40 watts, this will result in about 0.2 J of energy delivered into the treatment area, or about 0.2 J/cm² average fluence. Using a lens that renders the output beam non-uniform, delivering relatively high intensity spaced-apart central regions at 1 mm center-to-center distances surrounded by low intensity background regions, in such device, there are about 115 discrete subzones (e.g., combined areas of relatively high and relatively low intensity) per square centimeter in that arrangement, which results in about 1.73 mJ delivered to each subzone. Within each subzone, if the high intensity spaced-apart central region is about 120 μm in diameter and approximately 80% of the energy is delivered into the high intensity spaced-apart central regions, then the fluence within each high intensity region is approximately 12.2 J/cm². That fluence value in the device is comparable to the treatment fluence delivered by “professional” high-powered diode and Alexandrite uniform spot lasers having pulse durations of about 5 ms, which are the systems commonly used to treat uneven skin pigmentation in clinical settings by medically trained professionals. Unlike uniform-beam devices, using the non-uniform beam technology for any individual treatment session, only a relatively small percentage of the irradiated skin surface is actually treated with high intensity light, and thereby only a subpopulation of melanocytes receive a cellular disruptive dose of thermal energy, leading to a relatively smaller percentage of melanocyte damage per treatment area compared to uniform beam treatments. This advantageously reduces any sharp boundaries between treated and untreated skin, thereby reducing the need for special operator skills and techniques.

Table 1 lists examples of achievable values for the fluence in the relatively high intensity spaced-apart central regions of a non-uniform output beam device, when the output from a 40 W device is delivered in various spot diameters and various high intensity spaced-apart central region diameters. In addition, the average fluence (F avg) that the device would deliver to the skin in the absence of the diffractive lens array (e.g., with a uniform beam configuration) is also illustrated. For calculation purposes, the device on-time is assumed 5 ms and the lenses are assumed to deliver 60% of the incident radiation into the high intensity spaced-apart central regions, a more conservative value than that given in the example above.

TABLE 1

| | | High intensity fluence J/cm², | | |
|---|---|---|---|---|
| | | for spaced-apart central | | |
| Spot diameter, | | region diameter, μm | | |
| mm | F avg, J/cm² | 100 | 120 | 150 |
| 5 | 1.02 | 61.1 | 42.4 | 27.2 |
| 6 | 0.71 | 42.4 | 29.5 | 18.9 |
| 7 | 0.52 | 31.2 | 21.7 | 13.9 |
| 8 | 0.40 | 23.9 | 16.6 | 10.6 |
| 9 | 0.31 | 18.9 | 13.1 | 8.4 |
| 10 | 0.25 | 15.3 | 10.6 | 6.8 |
| 11 | 0.21 | 12.6 | 8.8 | 5.6 |
| 12 | 0.18 | 10.6 | 7.4 | 4.7 |

Good cosmetic effects can be produced by such non-uniform irradiation of tissues, due to differential effects occurring in both the relatively high intensity spaced-apart central regions of the beam and in the relatively low intensity background region. By way of illustration, within the spaced-apart central regions it is possible to cause relatively localized heating of tissues therein to a temperature T1 sufficient to heat up the melanocytes to a temperature sufficient to disrupt cellular processes, impair their function and decrease their pigment output, while simultaneously within the low intensity background regions at a lower relative temperature T2, collagen production is induced without causing undesirable thermal effects to the treated tissue within the background regions. The result of such treatment is an improvement to both skin texture and coloration. Other differential effects on tissues can be realized as well. By way of further example, within the spaced-apart central regions it is possible to cause relatively localized heating of tissues therein to a temperature T1 sufficient to remodel collagen structures, while simultaneously within the low intensity background regions at a lower relative temperature T2, collagen production is induced without causing undesirable thermal effects to the treated tissue within the background regions.

As described above, by decreasing the amount of energy delivered by the beam it is possible to select for specific thermal effects on tissues. For example, in our U.S. Pat. No. 7,856,985 we disclose collagen remodeling at temperatures where T1 is approximately 70 degrees C. or greater while the irradiated tissues in the cooler regions of the beam (e.g., at temperature T2) are not substantially adversely affected. The device used generating a non-uniform beam output, permits more selective application with less collateral tissue damage. However, for reducing age spots and evening skin pigmentation, melanocyte cell membrane damage with consequent cellular disruption is achieved at lower T1 temperatures of approximately 45-50 degrees C. (unless such heating is quite transient). Higher temperatures are suitable, and cause permanent disruption of melanocytes, but above 50 degrees C. more extensive thermal effects are seen in the tissue, that must be evaluated against therapeutic benefits. Below the temperature threshold for causing cellular damage and disruption, positive effects on skin tone are seen. At a T1 temperature of less than about 50 degrees C., cells are not substantially damaged but are still induced to generate a healing response, and express elastin, procollagen, keratin and other markers for dermal rejuvenation. So a device generating regions capable of elevating tissue temperatures to a T1 of about 45 degrees C. against a background T2 of about 37 degrees C. provides for tissue rejuvenation without substantially adverse thermal effects seen with “professional” high output spot beam laser systems.

Cellular damage from thermal effects begins to manifest at temperatures of about 40 degrees C. or greater depending partly on an individual's sensitivity. Erythema is one marker for treated skin, where the beam energy is sufficient to cause thermally-induced effects within at least the spaced-apart central regions. Visually observable erythema at a treatment site is correlated with parakeratosis and perivascular mononuclear infiltration (generally within or proximal to the spaced-apart central regions), both markers for microthermal injury to tissues. But it is possible to effectuate cellular responses without causing erythema. There are a number of detectable metabolic changes observed within treated cells even where T1 temperatures are only slightly in excess of about 37 degrees C. and where there is no observed erythema, for example, procollagen induction can be detected in such treated tissues as based on e.g., immunohistochemical analysis.

The overall effect of treatments on skin tone, wrinkling and pigmentation provides the best indication of therapeutic efficacy, but such treatments leave histological evidence that can be discerned. At higher energies, thermal damage is easy to detect. For more moderate energies, microthermal damage can produce effects that are seen with magnification although erythema provides a good marker for microthermal injury and it does not require microscopic examination of tissues from the treatment site. Generally, in the absence of any visually observable erythema, the cellular effects will be more subtle, or may take longer to manifest themselves or may require multiple treatments before visual improvement of the skin is seen. At lower output energies, shorter pulse durations, and longer intervals between treatments, it is advantageous to use more sensitive techniques to assay for cellular changes. While some of these may be more invasive in that they require a sample of the irradiated tissues (e.g., punch biopsies), these permit more sensitive and precise analysis of the cellular responses, these include for example, detection of mRNA by Northern blot or detection of protein by Western blot techniques. However, certain techniques provide for quantitative analysis, which can be correlated to describe a dose-response relationship for the non-uniform beam, as it is used in dermal rejuvenation applications. Such techniques include RT-PCR and/or real-time PCR, either of which permits quantitative measurements of gene transcription, useful to determine how expression of a particular marker gene in the treated tissues changes over time.

In addition to nucleic acid-based techniques, quantitative proteomics can determine the relative protein abundance between samples. Such techniques include 2-D electrophoresis, and mass spectroscopy (MS) such as MALDI-MS/MS and ESI-MS/MS. Current MS methods include but are not limited to: isotope-coded affinity tags (ICAT); isobaric labeling; tandem mass tags (TMT); isobaric tags for relative and absolute quantitation (iTRAQ); and metal-coded tags (MeCATs). MeCAT can be used in combination with element mass spectrometry ICP-MS allowing first-time absolute quantification of the metal bound by MeCAT reagent to a protein or biomolecule, enabling detection of the absolute amount of protein down to attomolar range.

Many genes and proteins are usable as markers for determining the dose-response of treated tissues, and currently preferable markers will show upregulation of gene expression (and protein) in connection with cellular growth and/or metabolic activity, with relatively lower levels exhibited by quiescent cells. For example and without limitation: heat shock proteins (HSP) are produced by cells in response to thermal stress and in conjunction with active metabolic processes, and as such provide for markers indicative of thermal effects on tissues. Matrix metalloproteinases (MMP) provide yet another marker indicating metabolic activity within dermal tissues. Keratin expression is also indicative of dermal rejuvenation. Procollagen can be detected in tissues following such treatments, as described in Example I, below. Many other cellular markers are described in the medical literature, that are indicative of dermal growth and enhanced metabolism, and the choice of marker is not intended to limit the scope of the invention described.

### EXAMPLE I

### Dermal Rejuvenation Using a Light-Based System

The following describes a light-based system used for dermal rejuvenation applications, e.g., reducing the appearance of fine lines and wrinkles, and reducing the visible effects of photodamage. We sought to determine the optimal laser exposure range that accomplishes three critical outcomes: 1) defines an exposure that generates an acute erythematous response, 2) defines an exposure that is suitable for daily use while being relatively pain-free or generating at least a tolerable pain sensation given the devices' use parameters, and 3) achieves these exposures with affordable laser sources thereby providing for a low-cost consumer device for home-use.

### Study Design

In the initial study, 14 subjects including 2 males and 12 females having an age range of 41 to 58 years (avg. 47 years) were exposed to laser energy in the form of a non-uniform output beam, at a region on their lower back, using a range of power settings. Subjects ranged from Fitzpatrick skin type 1 to IV with the majority of subjects of skin type II (8 subjects). Subjects were graded using Unilever Photodamage/Irritation Scale Rev. 91709, by an Expert Grader and study subjects presented with Fine Line & Wrinkle grades from Mid-Moderate (Grade 5) to Low-Severe (Grade 7). Subjects were confirmed not to have a history of excessive smoking, sun exposure or use of tanning beds.

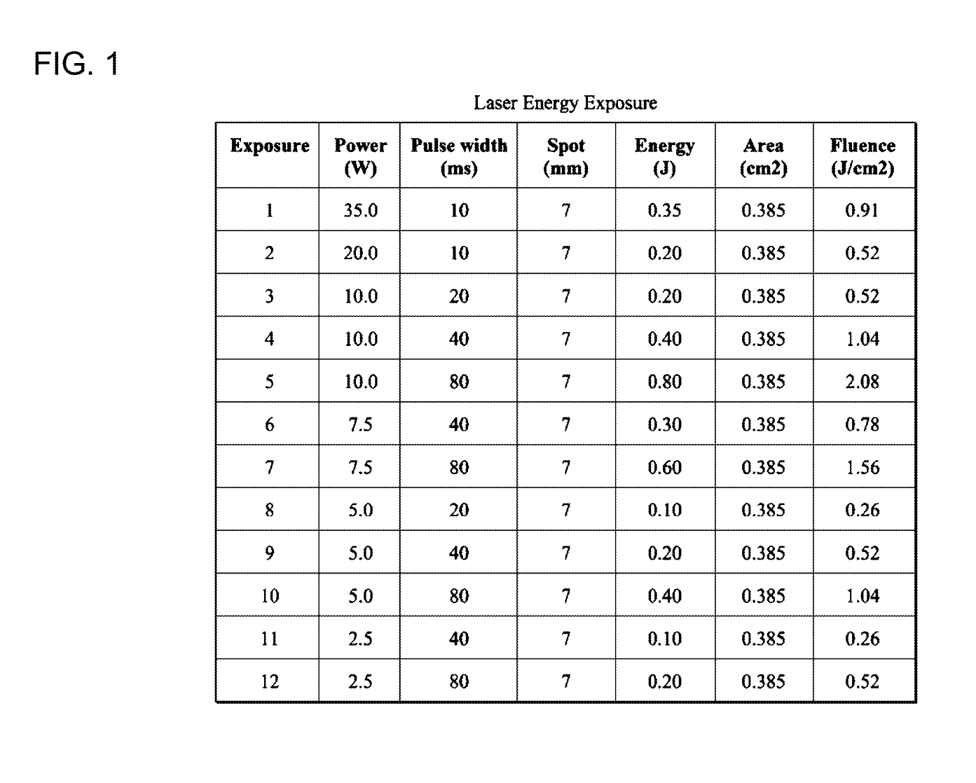

Using the energy values provided in FIG. 1, patients received 20 cumulative exposures on their lower back in 12 distinct zones, the exposures being once per day, each weekday (e.g., 5 consecutive days of treatment followed by two consecutive days of no treatment) for four weeks, followed by a 6 week no-treatment regression period. A template was used each day to orient the treatment zones to ensure proper repeat dosing. Each corresponding treatment zone therefore represents a particular power setting and exposure time. Visual observations, photographs and Mexameter readings were obtained both pre and post daily treatments.

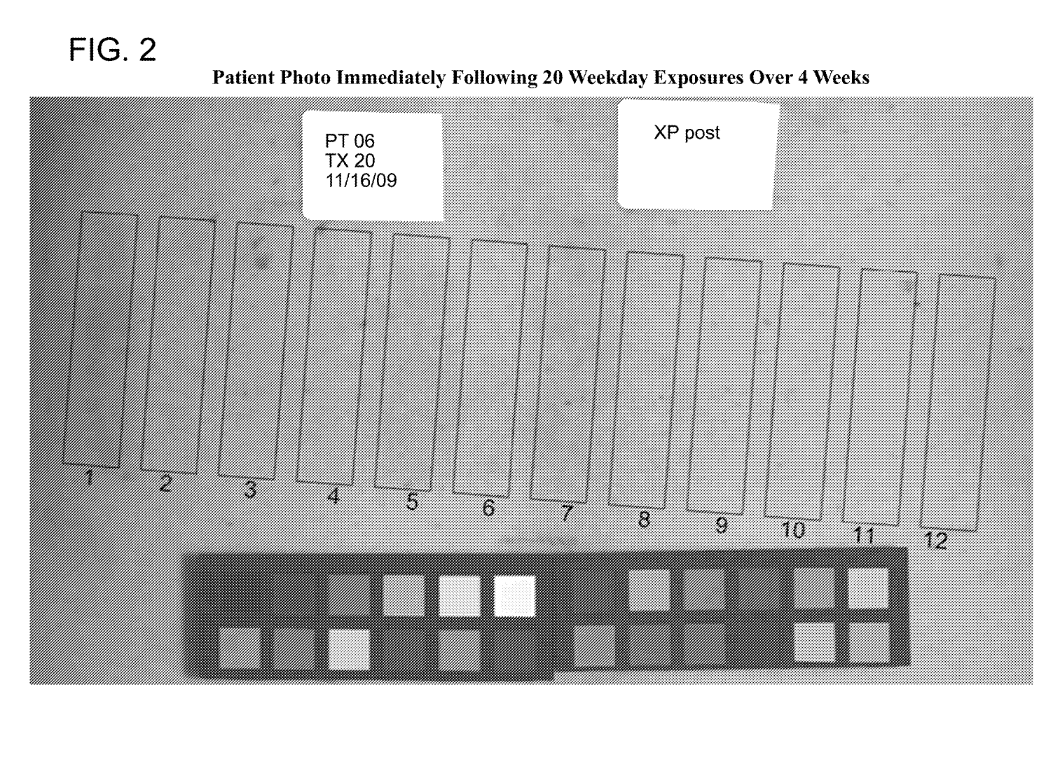

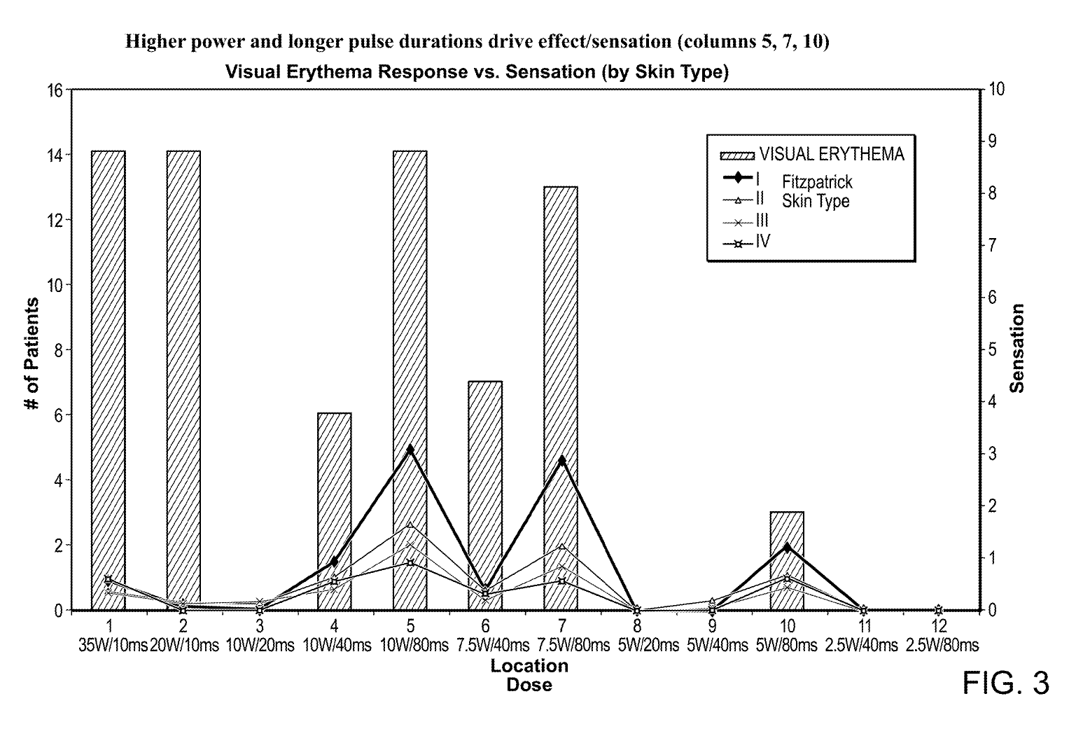

FIG. 2 illustrates the response of one patient to irradiation, which exemplifies the general responses of patients in the study. Erythema is observed in columns #1, #5 and #7. Column #1 represents a positive control which was matched to the lowest possible energy settings provided by Cynosure's current “professional” laser systems. It was expected that virtually all patients should produce an acute clinical response (erythema) from exposures provided in this column. Columns #5 and #7 are doses with a power setting of 10 watts and 7.5 watts respectively with matched exposure durations of 80 milliseconds. Visual notations of erythema in the respective columns demonstrated a clear pattern that longer exposure times in combination with higher power settings increased the intensity of response across all patients. In FIG. 3, it is noted that both sensation and visual clinical response increased in columns with higher power and exposure durations in combination.

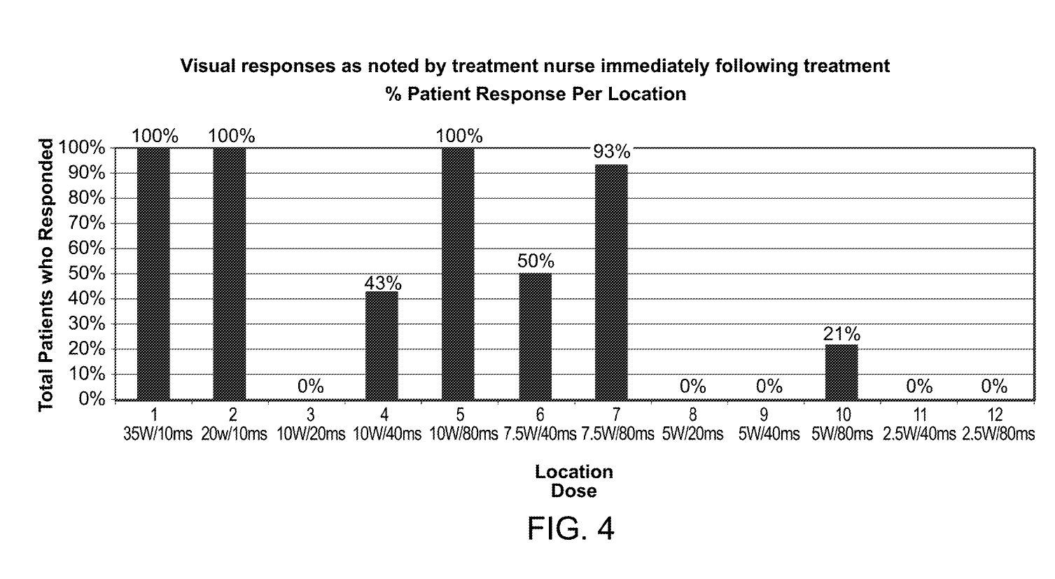

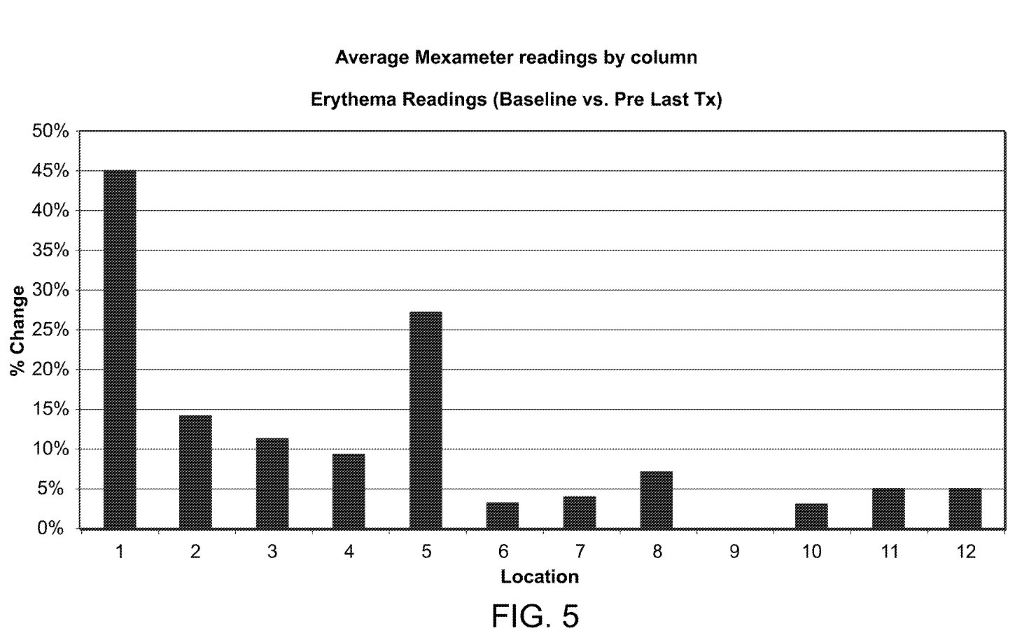

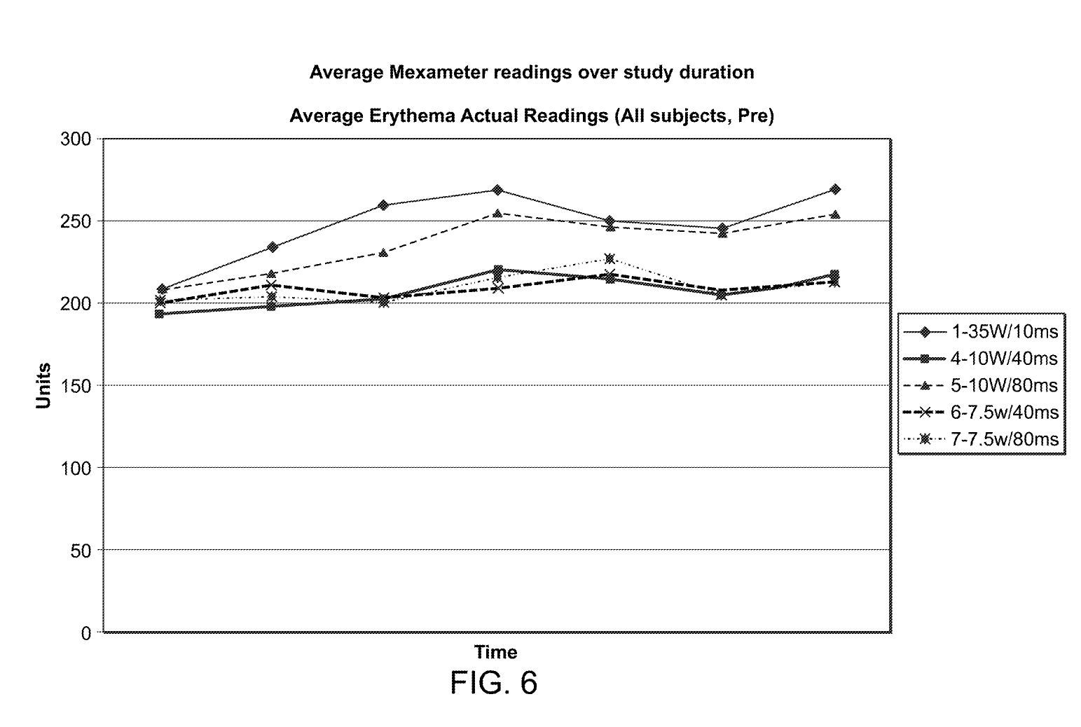

FIG. 4 demonstrates that columns #1, 2, 4, 5, 6, 7 & 10 produced a visual clinical response. Columns #1 & 2 featured power settings that are efficacious, but are outside of the desired power range for of a consumer-based device. Out of the remaining columns, it is noted that exposure durations at 80 msec. in combination with power settings above 7.5 watts produced a pronounced clinical effect as well as an increase in sensation. Power settings below 5 watts with any exposure setting produced virtually no visible or measured clinical response. Mexameter readings, shown in FIG. 5 are consistent with the above visual responses. In addition, average Mexameter readings over the duration of the study shown in FIG. 6 indicate the therapeutic response is cumulative. The initial study provided guidelines for establishing appropriate parameters for the Phase Two study based on the variables of time and energy. Energy settings or exposure durations alone are not the driving factor behind clinical response, and their combination drives clinical effects.

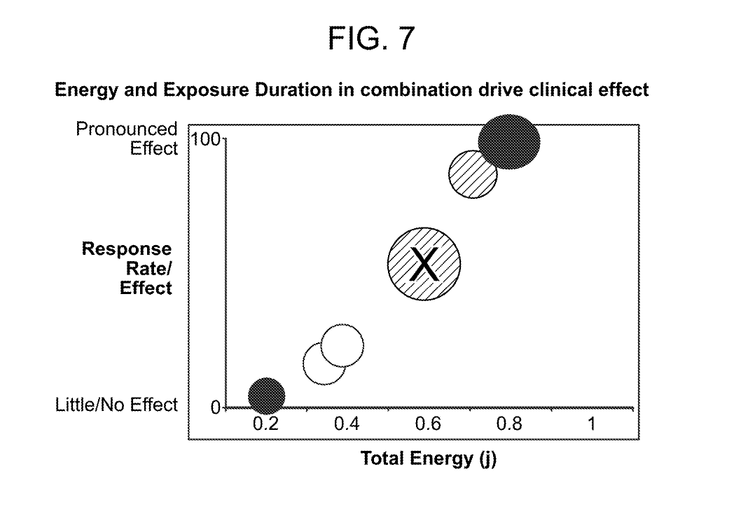

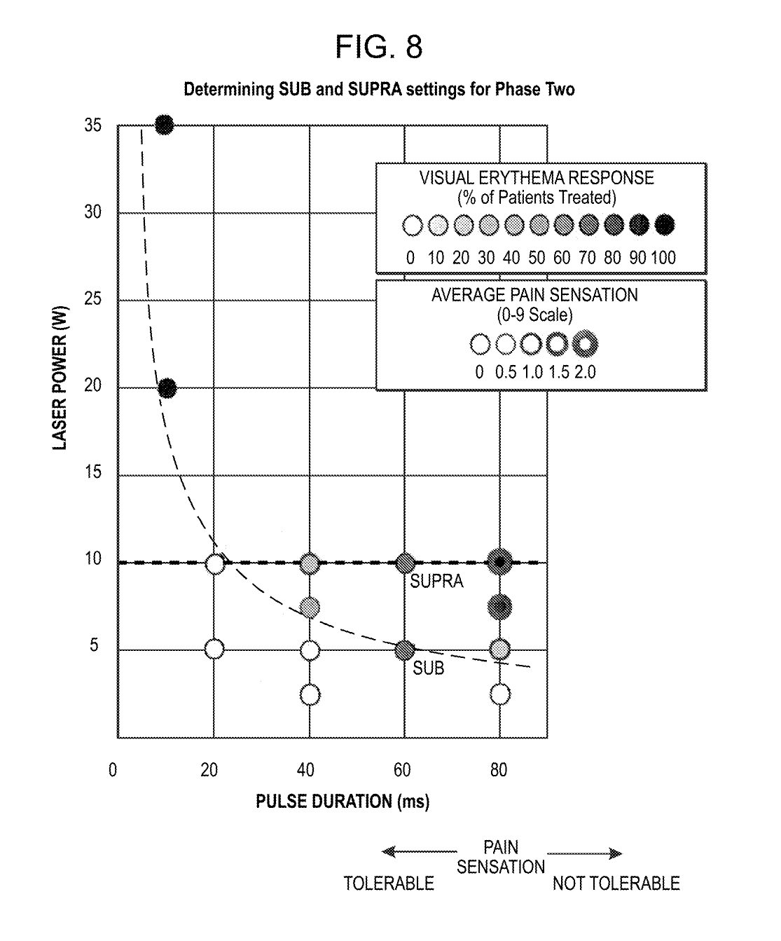

For phase two of the study, in determining the SUB and SUPRA settings, we analyzed the individual parameters that produced clinical responses in the skin. At energy settings of 0.8 joules (10 W outpower and 80 msec pulse duration), subjects demonstrated acute clinical responses and sensation scores increased. Conversely, at energy settings of 0.4 joules (10 watts and 40 msec), subjects demonstrated reduced sensation but less pronounced clinical effect. As shown in FIG. 7, for the phase two study we selected an energetic value of 0.6 joules, where we observed a relatively consistent clinical response, with lower sensation scores. At power settings below 5 watts, there was decidedly no clinical effect observed visually at any exposure duration. See FIG. 8. However, there are cellular effects at such power settings, that are not visually observable but can be detected with the more sensitive assays described above, but at such energy settings the clinical effects are subtle and prolong the required treatments.

In order to maintain a safe and consistent exposure to facial tissue, it was decided that, in view of our objectives for a consumer-operated facial treatment system, 10 W delivered in an 80 msec pulse produced a clinical effect that represented the desired upper limit of treatment pulses, and 5 W delivered in a 40 msec pulse produced a minimal clinical effect which represented the desired lower limit for treatment pulses. A fixed 60 msec pulse duration approximated the best combination of tolerable sensation, visual erythema and observed positive clinical effect on tissues, when the power was modulated between 5 watts (SUB) and 10 watts (SUPRA). See, FIG. 8. Differentiation of clinical effect in treatment subjects was observed between the SUB and SUPRA settings.

Using these settings, fourteen subjects each received a daily facial treatment over twenty weekdays, at 2 power settings SUB (5 W, 60 msec) and SUPRA (10 W, 60 msec) administered respectively on each side of the face using a 1470 nm, CAP (Combined Apex Pulse) array laser device, which generated an output beam characterized by an array of relatively small, relatively high-intensity, spaced-apart central regions superimposed on a relatively large, and relatively low-intensity background region.

Subjects were randomized and balanced for age and wrinkle severity. Facial exposures were over a 4 week treatment period followed by a 6 week regression period. Subjects were evaluated at baseline, end of treatment and end of the regression period, using a high resolution Canfield photography system and silastic impression molds of the skin in the treatment area, and erythema levels were established using a Mexameter. Following 4 weeks of daily exposures, subjects were evaluated to determine the presence of erythema and any signs of improvement in fine lines and wrinkles or photodamage markers. Unilever Expert Graders evaluated subjects at five time points during the study: 1) baseline; 2) prior to 20^{th} treatment [pre 20]; 3) 20 minutes post treatment [post 20]; 4) 24 hours post 20^{th} treatment [post 20_{—}24hrs] and 5) following the 6 week regression period [6 wk post_tx20]. To demonstrate progressive healing effects a subset of 3 study subjects were identified to receive an additional set of 2″×2″ of CAPs modified laser exposures in a small, photo-exposed area on the back for biopsy purposes. These areas were treated in the exact manner as their facial treatments. Histology samples were taken by punch biopsy at Day 0 (control), Day 1 (immediately post last treatment), Day 3, Day 7 and Day 14 day following 20 laser treatments. An additional biopsy was obtained at Day 60 to be stained specifically to identify markers for procollagen I, elastin and decorin.

### Results

Erythema

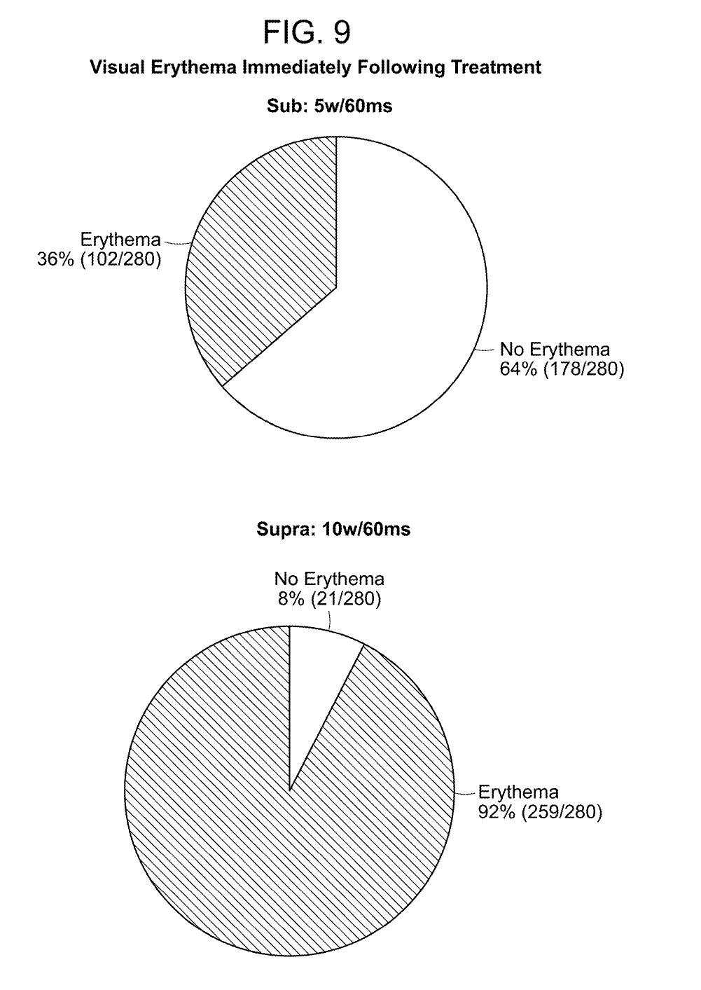

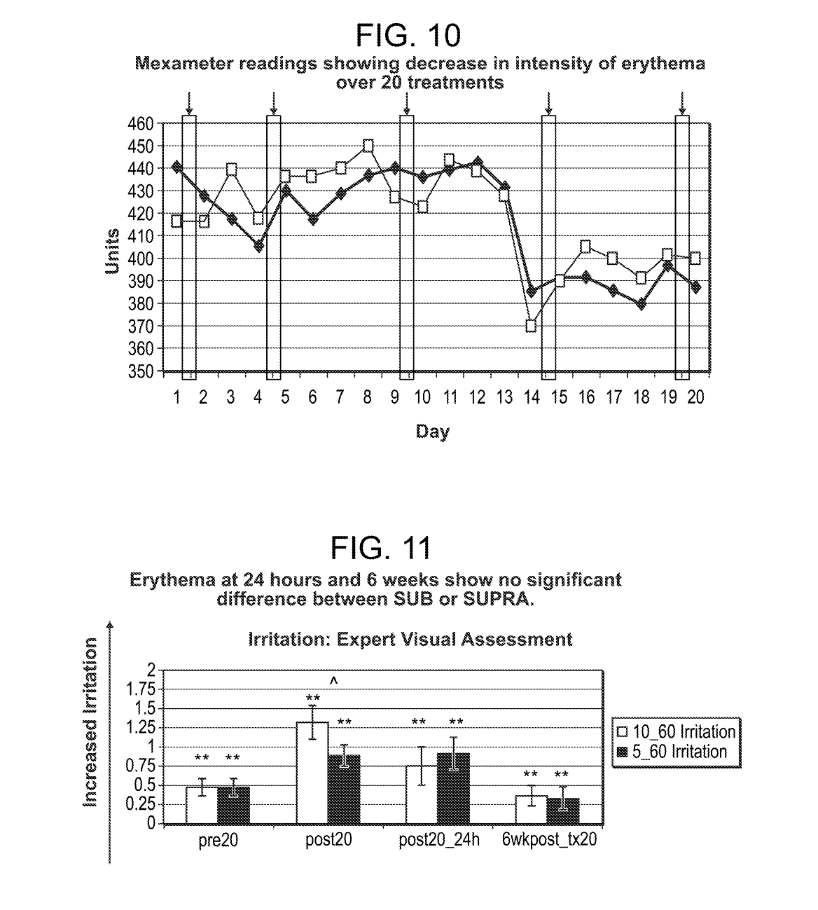

Erythema scores were recorded both visually by the nurse technician and additionally using the Mexameter both prior to and immediately post treatment. A series of 3 readings were taken in the treated areas (SUB and SUPRA) each day immediately following treatment. The average of these scores was recorded in the daily patient chart. Expert Graders also recorded grades for irritation using the Unilever 4-point Erythema grading scale at the 5 measurement time points during the study. In the SUB category (5 W/60 msec.), only 36% of subjects demonstrated visible erythema immediately following treatment as recorded by the nurse technician. In the SUPRA category (10 W/60 msec.), 92% of subjects demonstrated visible erythema immediately following treatment. See, FIG. 9. When comparing Mexameter readings of immediately post treatment versus pre treatment, subjects demonstrated a slight decrease in intensity of erythema from treatment over the course of 20 treatments. The decrease of erythema in the SUB area over 20 treatments was 11% and the decrease in the SUPRA area was 9%. See, FIG. 10.

In addition to the visual grading by the Nurse technician and the Mexameter, Expert Graders were used to assess erythema/irritation. Expert grader sessions were approximately 10-15 minutes following actual laser treatment. As expected, Expert grader scores demonstrated elevated levels of erythema immediately following treatment in both SUB and SUPRA areas. Erythema in the SUPRA area was more pronounced than in the SUB area immediately following treatment. At the 24 hour time point, erythema levels in the SUPRA area decreased dramatically and showed no appreciable difference versus SUB areas. No difference in SUB / SUPRA erythema scores were noted at the 6 week follow-up. See, FIG. 11.

Melanin

Melanin (pigment) scores were also recorded using the Mexameter prior to and immediately following laser treatment. A series of 3 readings were taken in the treated areas (SUB and SUPRA) each day immediately following treatment. The average of these scores was recorded in the patient charts by the nurse technician. It is noted that the melanin scores in both the SUB and SUPRA areas dropped by 11% over the course of 20 sequential treatments spanning 4 weeks. FIG. 12 demonstrates the respective average drop in melanin scores as recorded by the Mexameter. The duration of this study occurred during a time of the year when melanin is typically fairly stable as opposed to other times during the year when sun exposure is increasing (spring/early summer) or decreasing (late summer/early fall).

Sensation

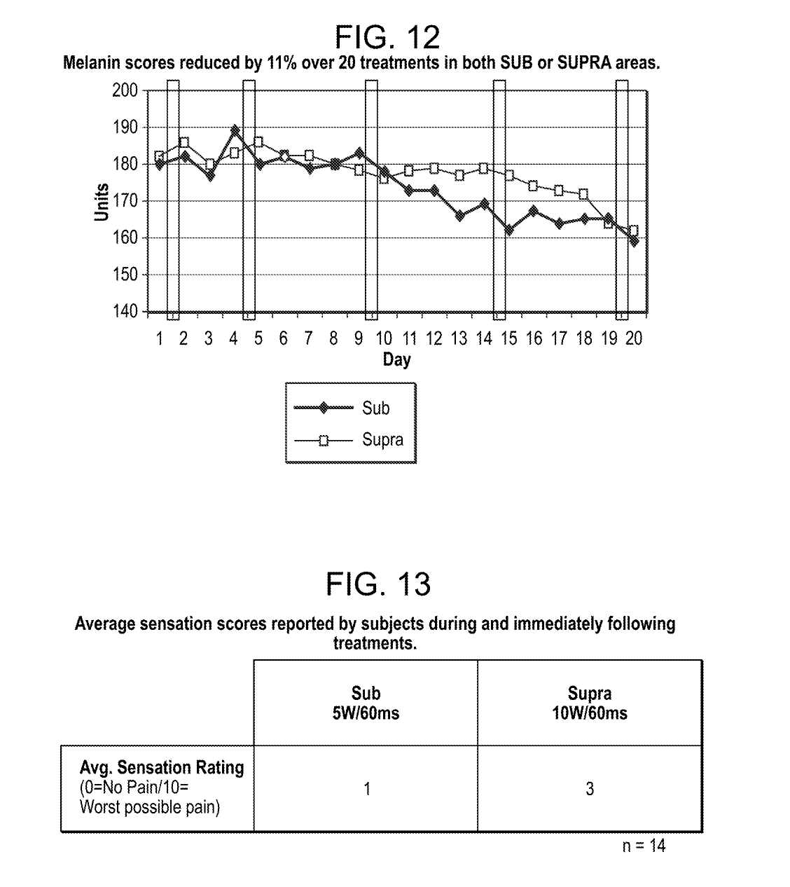

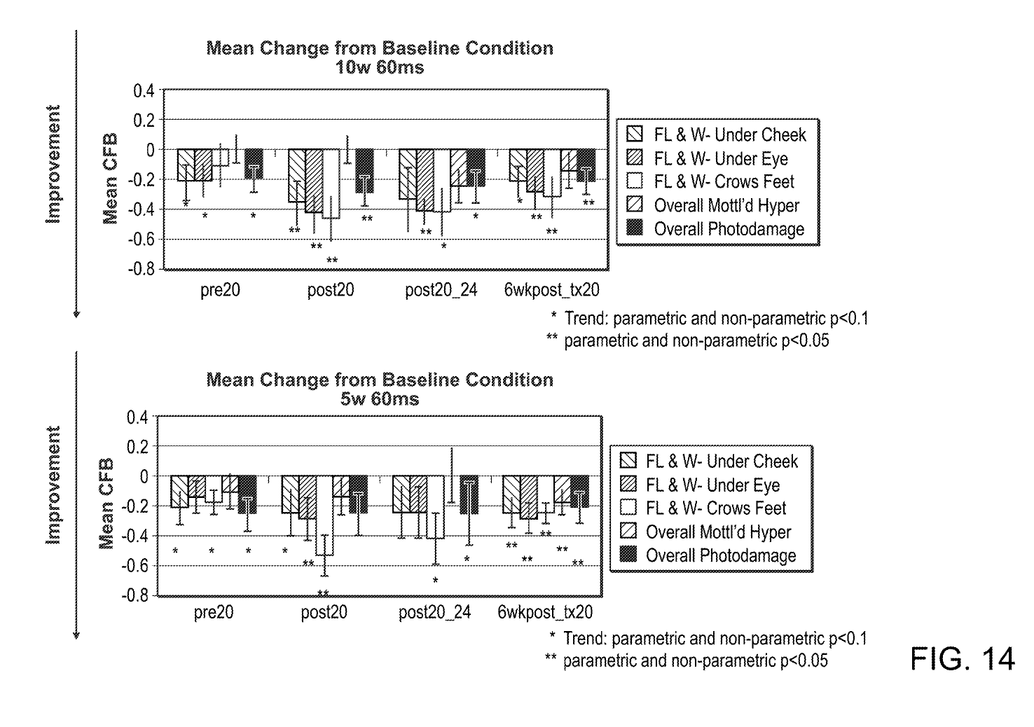

Patients were queried regarding sensation both prior to and immediately following treatment. A 10 point visual scale was used by patients to rate the sensation that they were experiencing. Patients did not report any lingering pain remaining from the previous treatments. On average, patients rated the sensation immediately following treatment to be a 1 (out of 10) in the SUB area and a 3 (out of 10) in the SUPRA area. This did not vary widely based on age or skin type. Regionally, patients commented that the sensation increased near the eye (in both crow's feet and under eye regions). If patients commented about increased sensation in these areas, the nurse would ask them to rate the sensation using the existing chart. On average, when subjects commented about increased sensation in these areas, they rated the sensation to be a 2 (out of 10) in the SUB area and a 4 (out of 10) in the SUPRA area. See, FIG. 13.

Expert Grader Evaluations

Expert graders performed visual skin assessments on all subjects at baseline, midpoint of treatment period, end of treatment period and again at the end of the 6-week regression period. Expert graders used the 9-point Fine Line and Wrinkles/Photodamage grading scale supplied by Unilever. At the end of the treatment period, Expert Graders reported that all areas assessed showed significant improvement in fine lines/wrinkles in both the SUB and SUPRA areas. The magnitude of improvement in the under eye area at the end of the treatment period was greater in the SUPRA area versus the SUB area. Expert Graders also reported that improvements in overall photodamage scores improved in both SUB and SUPRA areas at the end of the treatments. At the end of the 6-week regression period, Expert Graders returned for the last grading session. The expert graders reported that the improvements in fine lines/wrinkles as well as the photodamage scores were maintained over the regression period. The magnitude of improvement in the crow's feet area at the 6-week follow-up was greater in the SUPRA areas when compared to the SUB. The under eye and crow's feet areas showed very good improvement as well. Photodamage scores showed strong improvement at the 6-week follow-up without any regression noted. See, FIG. 14. It should be noted that short-term inflammation and transient edema following laser treatment causes an immediate improvement of both fine line/wrinkle and photodamage scores. In the professional market segment this is often referred to as “flash edema” and is known to be offered as an aesthetic treatment just prior to social engagements.

Replica Molds/Primos Texture Measurements

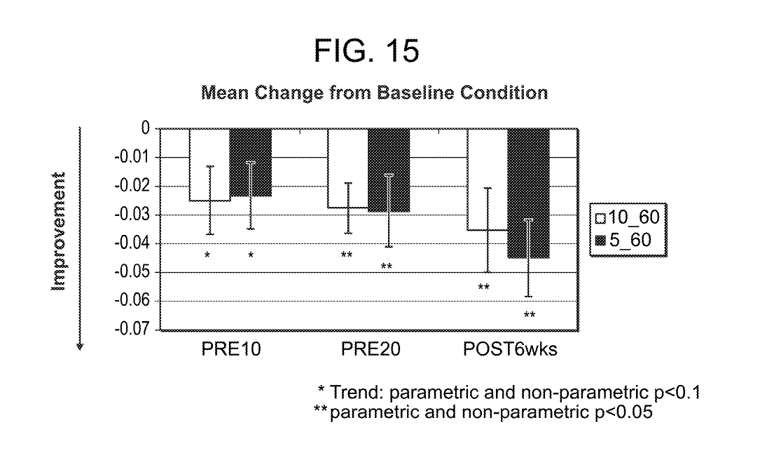

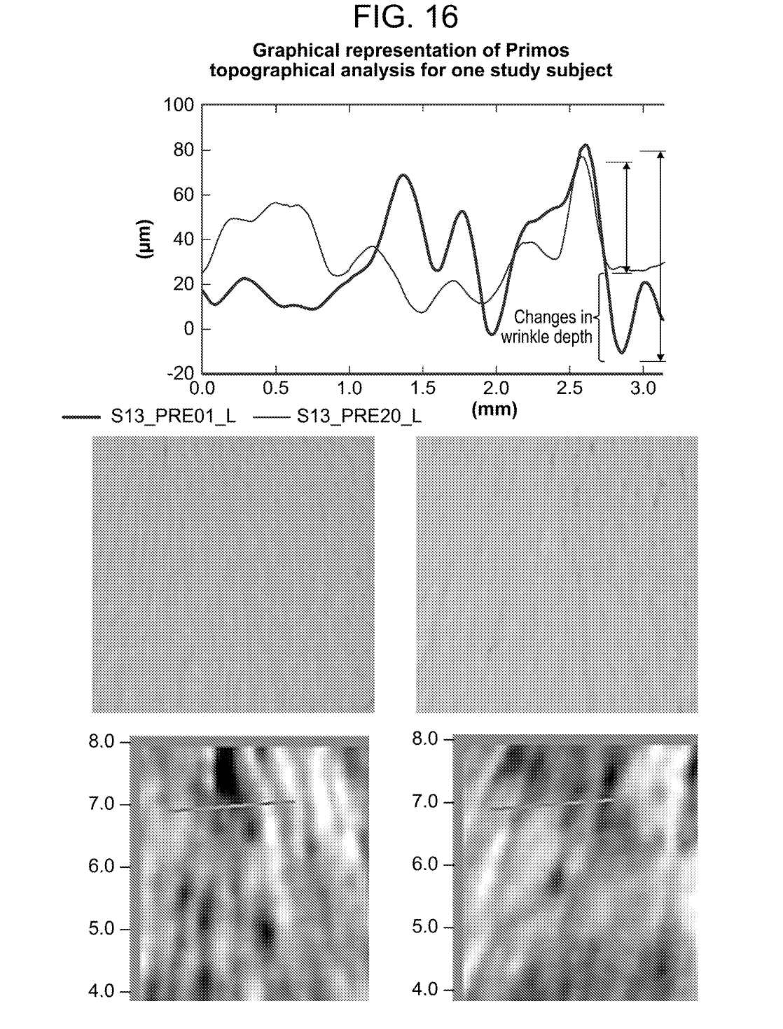

Replica molds, or negative impressions of the surface of the skin were obtained at baseline, treatment mid-point, end of treatment and end of regression period. Sample impressions were taken at specific locations in the crow's feet and middle-cheek area (lateral to nasal labial folds). Samples were analyzed by Unilever using the Primos computer topography system. The Primos system analyzes micro changes in the surface topography of the skin and provides a summary analysis of respective changes. In the analysis, the most prominent lines were chosen for measurement. At end of 20 consecutive, weekday treatments, a significant improvement to the length and depth of the wrinkles were observed. Improvements recorded in length and depth of wrinkles were maintained following the 6-week regression period. No significant differences were noted between the SUB and SUPRA areas. No appreciable changes in the roughness or texture scores were reported. See, FIG. 15. As part of the Primos topographical surface analysis both 2D and 3D surface images were obtained for each subject. A comparison versus baseline was measured for all patients at each time point. Sample Primos images for one exemplary subject are noted in FIG. 16.

Subject Response to Treatment

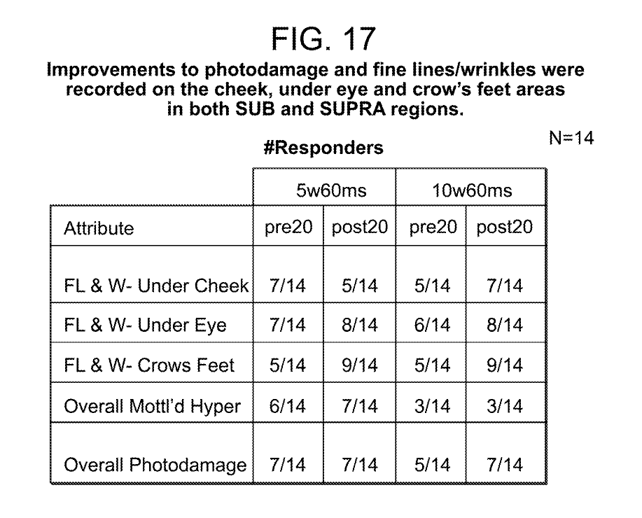

The rate of response was recorded by treatment areas and segmented across SUB and SUPRA treatment areas. Improvements to photodamage and fine lines/wrinkles were recorded on the cheek, under eye and crow's feet areas in both SUB and SUPRA regions. A greater response rate was noted in both the crow's feet and under eye regions. See, FIG. 17.

Biopsy Analysis

Biopsies were obtained to demonstrate a progressive healing response. A subset of 3 (out of 14) subjects were identified to receive an additional laser treatment area (approx. 2″×2″) in a photo-exposed area of the back. These areas were treated in the exact manner as the SUB and SUPRA facial treatments. Biopsy samples (2mm punch) were taken at Day 0 (control), Day 1 (post the last treatment), Day 3, Day 7 and Day 14 post 20 laser treatments. An additional biopsy was obtained at Day 60 to be stained specifically to biomarkers for Pro-collagen I, Elastic and Decorin.

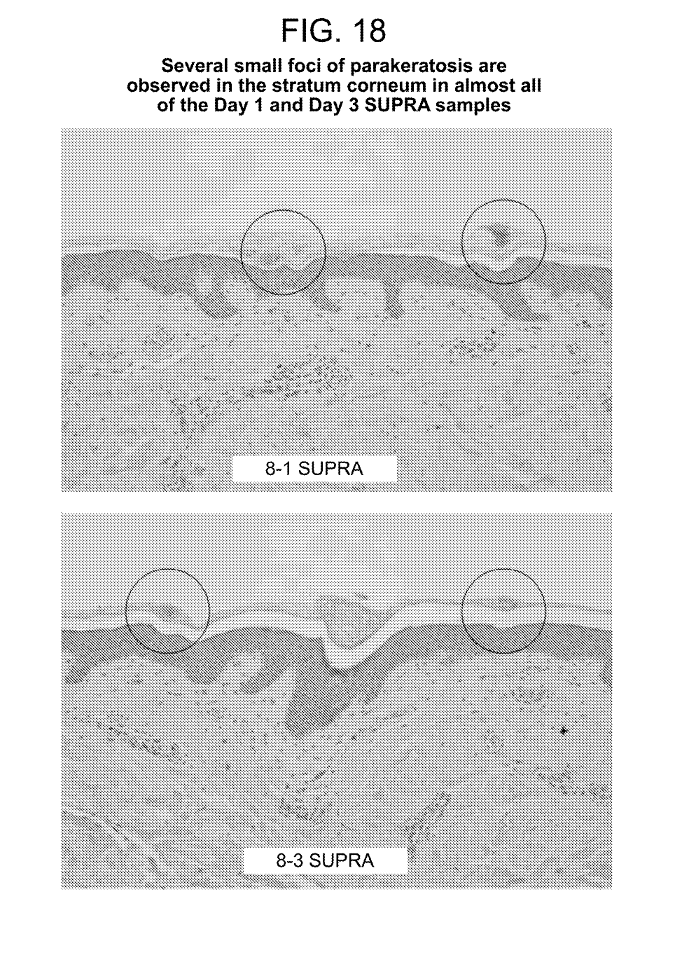

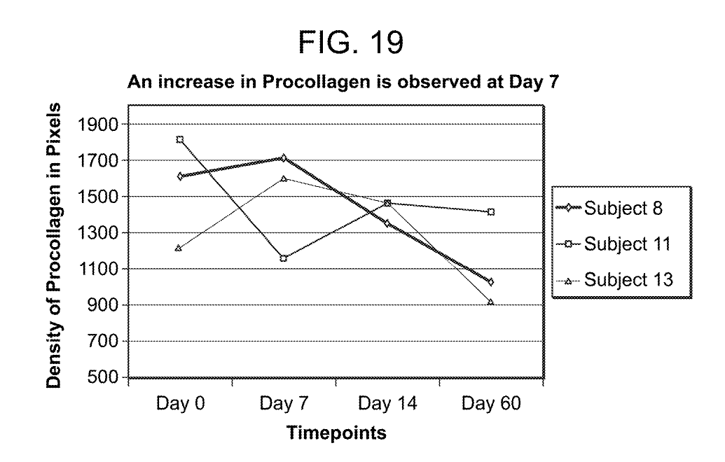

Pathologists reported that there were obvious changes in the SUPRA slides out to the Day 3 sample but all findings returned to normal by the Day 7 and Day 14 samples. No obvious abnormalities were noted in the SUB samples. Specifically, the pathologist report noted there were several small foci of parakeratosis in the stratum corneum in almost all of the Day 1 and Day 3 SUPRA samples. Within the foci, there appear to be areas of prior keratinocyte necrosis. The parakeratosis noted is a “shedding of cells” most commonly seen from a prior thermal injury which is consistent with micro thermal injuries associated with fractionated laser exposures. See, FIG. 18. The dermis also contains mononuclear infiltrate and scattered melanophages. In one small focus in the dermis, there appears to be collagen denaturation adjacent to perivascular mononuclear infiltrate. Epidermal thickening and enhanced dermal fibroblast proliferation in the SUPRA treatment sites were seen in all biopsy subjects. Parakeratosis was noted in all SUPRA samples from Day 1 to Day 3 in all subjects. All findings returned to normal in the Day 7 and Day 14 samples. For two of the subjects there was an increase in the density of Procollagen at Day 7 for the SUPRA setting and then a decrease in density for the Day 14 sample and beyond. See, FIG. 19.

Photographic Analysis

High resolution photographs for all subjects were analyzed to identify improvements in skin rejuvenation and reduction of visible photodamage effects. While photographic review is possible, it is a qualitative approach subject to differences in the patient's skin (shine) and the camera (lighting) on a daily basis. In this study, all subjects were photographed using a Canfield High Resolution photographic system (Model: Visia-CR) in order to standardize position, lighting and other effects that could affect the consistency of the photography. Subjects were photographed at baseline, pre/post treatment every 5 days, end of treatment cycle and end of regression period. Improvements in fine lines/wrinkles and photodamage were noted in the study subjects.

Study Conclusions

Study data was very positive for laser phototherapy across all key measurement criteria. Expert graders reported improvements in fine line/wrinkles scores in both SUB and SUPRA categories which were maintained after the 6-week regression period. Fine lines and wrinkles improved most markedly in the crow's feet and under eye regions when compared to other regions. It should be highlighted that the regression period was longer than the actual treatment duration. Currently, to our knowledge, there is not a topical therapy on the market that can generate improvements in fine line/wrinkles and photodamage categories following 4 weeks of therapy and also maintain clinical benefits after a 6-week regression period.

With respect to the differences between SUB and SUPRA, the SUPRA settings caused greater short-term erythema. Erythema levels in the SUPRA regions matched the SUB regions at the 24 hour time point and were well within acceptable ranges. Increased sensation was not a major factor for patients in the SUPRA region when compared to the SUB region. The SUPRA settings may have also produced more significant and lasting benefits at the 6-week follow-up in the crow's feet region. Histological benefits were also in favor of the SUPRA settings, demonstrating evidence of more pronounced microthermal effects in the SUPRA category when compared to the SUB category. It should also be noted that the greater clinical effect of the higher SUPRA settings aligns well with the increased clinical effect seen at greater power settings in the professional market segment.

### Equivalents

Those skilled in the art will recognize, or be able to ascertain, using no more than routine experimentation, numerous equivalents to the specific compositions and procedures described herein. Such equivalents are considered to be within the scope of this invention, and are covered by the following claims.

We claim:

1. A dose determination method for dermal rejuvenation, comprising the steps of:
A. illuminating dermal tissue of a human with one or more pulsatile doses of therapeutic monochromatic light having a wavelength λ delivered to the dermal tissue as a non-uniform beam, wherein the non-uniform beam is characterized by a cross-section corresponding to an array of relatively small, relatively high intensity, spaced-apart central regions superimposed on a relatively large, relatively low intensity background region; wherein each pulsatile dose is characterized by an intensity and a duration sufficient to cause detectable expression of markers in the dermal tissue;
B. detecting in the dermal tissue expression of cellular markers induced by the pulsatile dose; and
C. for each pulsatile dose of step A, determining the paired values of intensity and duration of the dose with detection of the induced cellular markers.

2. The method of claim 1, where detection of cellular markers is quantitative.

3. The method of claim 2, where the paired values of intensity and duration of the dose are correlated with quantitative detection of cellular markers to derive a dose-response curve.

4. The method of claim 1, where the cellular markers in the dermal tissue comprise keratinocyte polypeptides.

5. The method of claim 1, where the cellular markers in the dermal tissue comprise chondrocyte polypeptides.

6. The method of claim 3, where the chondrocyte polypeptides comprise procollagen.

7. The method of claims 1, where the cellular markers are detected at higher levels relative to non-illuminated dermal tissue.

8. The method of claim 1, where the dose of therapeutic light is cumulative, and delivered in a plurality of exposures.

9. The method of claim 1, where the dose of therapeutic light is sufficient to cause erythema in the dermal tissue, but does not cause substantial pain in the human.

10. The method of claim 1, where the dose of therapeutic light is sufficient to cause heating of tissue in the relatively high intensity spaced-apart central regions of the beam to a temperature sufficient to cause microthermal damage, but does not cause substantial heating of tissue in the relatively low intensity background region of the beam.

11. The method of claim 10, where cells in the tissue within the relatively low intensity background region of the beam are induced to express one or more detectable markers indicative of metabolic activity.

12. A method for treating photoaging of human skin, comprising:
generating an output beam from a laser source;
coupling the output beam into an optical system that modifies the output beam to provide a treatment beam having a non-uniform energy profile, said non-uniform energy profile being comprised of regions of relatively high energy per unit area within a substantially uniform background region of relatively low energy per unit area in comparison to the regions of relatively high energy per unit area; and
directing the treatment beam to a target tissue area characterized by hyperpigmentation such that the regions of relatively high energy per unit area of the beam illuminate portions of the target tissue and deliver sufficient energy to such portions of the target tissue to heat select such portions of the target tissue to a first temperature T1, and wherein the substantially uniform background region of relatively low energy per unit area of the treatment beam illuminates the remaining portion of the target tissue and delivers sufficient energy to such remaining portion of the target tissue to heat the remaining portion of the target tissue to a second temperature T2, T2 being less than T1; and
irradiating the treatment area such that a plurality of melanocytes in the regions of relatively high energy per unit area of the treatment beam receive a cellular disruptive disruptive dose of thermal energy, thereby attenuating melanin expression in the treated tissue.

13. The method of claim 12, wherein the treatment beam regions of relatively high energy per unit area heat select portions of the target tissue to a first temperature T1 of 45 degrees C. or higher.

14. The method of claim 12, wherein the laser source comprises a diode laser.

15. The method of claim 12, wherein the wavelength of the output beam is between about 500 nm and 1100 nm.

16. The method of claim 12, wherein the treatment beam at the target tissue area has a diameter between about 5 and 12 mm.

17. The method of claim 12, wherein the average fluence of the treatment beam at the target tissue in the regions of relatively high energy per unit area is less than about 13.0 J/cm2.

18. The method of claim 12, wherein the average fluence of the treatment beam at the target tissue area is less than about 1.0 J/cm2.

19. The method of claim 12, wherein the output beam has a pulse duration of between 0.1 and 100 milliseconds.

20. The method of claim 12, wherein the output beam has a pulse duration of between 5 and 60 milliseconds.

21. The method of claim 12, wherein the optical system comprises a diffractive lens array such that each lens in the array provides a region of relatively high energy per unit area, the regions of relatively high energy per unit area within a substantially uniform background region of relatively low energy per unit area in comparison to the regions of relatively high energy per unit area.

22. The method of claim 21, wherein the diffractive lens array comprises about 2000 or less lenses in the array.

23. The method of claim 22, wherein each lens is between about 150 and 1000 microns in diameter.

24. A system, comprising:
a laser source that generates an output beam;
and an optical system coupled to the output beam, the optical system modifying the output beam to provide a treatment beam having a non-uniform energy profile, said non-uniform energy profile being comprised of regions of relatively high energy per unit area within a substantially uniform background region of relatively low energy per unit area;
the treatment beam configured such that the regions of relatively high energy per unit area output sufficient thermal energy to heat target tissues illuminated within the regions of relatively high energy per unit area to a first temperature T1, and wherein the substantially uniform background region of relatively low energy per unit area outputs sufficient thermal energy to heat target tissues illuminated within the background regions to a second temperature T2, T2 being less than T1.

25. The system of claim 24, wherein the treatment beam regions of relatively high energy per unit area heat select portions of the target tissue to a first temperature T1 of 45 degrees C. or higher.

26. The system of claim 24, wherein the laser source comprises a diode laser.

27. The system of claim 24, wherein the wavelength of the output beam is between about 500 nm and 1100 nm.

28. The system of claim 24, wherein the treatment beam at the target tissue area has a diameter between about 5 and 12 mm.

29. The system of claim 24, wherein the average fluence of the treatment beam at the target tissue in the regions of relatively high energy per unit area is less than about 13.0 J/cm2.

30. The system of claim 24, wherein the average fluence of the treatment beam at the target tissue area is less than about 1.0 J/cm2.

31. The system of claim 24, wherein the output beam has a pulse duration of between 0.1 and 100 milliseconds.

32. The system of claim 24, wherein the output beam has a pulse duration of between 5 and 60 milliseconds.

33. The system of claim 24, wherein the optical system comprises a diffractive lens array such that each lens in the array provides a region of relatively high energy per unit area, the regions of relatively high energy per unit area within a substantially uniform background region of relatively low energy per unit area in comparison to the regions of relatively high energy per unit area.

34. The system of claim 33, wherein the diffractive lens array comprises about 2000 or less lenses in the array.

35. The system of claim 34, wherein each lens is between about 150 and 1000 microns in diameter.
